(19) United States
(12) Patent Application Publication
(10) Pub. No.: US 2020/0352767 A1
(43) Pub. Date: Nov. 12, 2020

(54) DEVICE AND METHOD OF WEIGHT CONTROL VIA IMPLANTABLE NET-SHAPED BASKET SYSTEM

(71) Applicant: Andrew Zhang, New York, NY (US)

(72) Inventor: Andrew Zhang, New York, NY (US)

(21) Appl. No.: 16/404,764

(22) Filed: May 7, 2019

(51) Int. Cl. A61F 5/00; A61B 5/03

(52) U.S. Cl. CPC A61F 5/0063; A61B 2562/028; A61F 2005/002; A61B 5/03

(57) ABSTRACT

A device for controlling the weight of a body comprises a net-shaped basket system. The basket is custom made for each patient by 3D printing technology using silicone material based on a physician's assessment of height, weight, and energy consumption of obese patients. With several MEMS pressure sensors embedded inside the basket material, it is placed on the outer wall of the patient's stomach by laparoscopic surgery. The sensor can measure the pressure of the stomach in real time. When the patient eats too much, the pressure reaches the threshold level set by the doctor, and the monitor will immediately remind the obese person to stop eating. If the patient ignores the warning and continues to eat, the basket will reach its distensible limit, forcing a patient to stop taking food. Food intake of obese patients can be controlled quantitatively via the basket system and the gastric motility can be diagnosed via the pressure-time curve.

### FIELD OF THE INVENTION

This invention relates to weight control, and more particularly to a device and method for controlling body weight via implantable net-shaped basket system.

### BACKGROUND

Obesity has been steadily increasing in the United States and worldwide. From the perspective of diet, obese patients have poor self-control in food intake, or due to the failure of the neurofeedback mechanism, they eat too much food, especially the high-fat diet, which is the main cause of obesity. When ingested fat gets into the bloodstream, some of it is oxidized to provide the body with the heat it needs to function. If eat too much, the quantity of heat that airframe place absorbs exceeds normal to use up, the quantity that the adipose in food enters person adipose bank to store can increase, form adiposity thereby. Approximately 500,000 people in North America and Western Europe are estimated to die from obesity-related diseases every year and obesity is estimated to affect more than one billion adults worldwide. There is a pressing and unmet need for a solution to the epidemic problem. [03] Various techniques have been known for reducing obesity in patients. One general category of obesity surgery targets the relative absorption of food. This type of procedure seeks to shorten the length of, or otherwise modify, the small intestine to limit the amount of foods that is ultimately absorbed by the body (malabsorption). Common examples of malabsorption procedures include: gastric bypass (e.g., Roux-en-Y gastric bypass); billiopancreatic diversion; and intestinal bypass. Other surgical methods address obesity via restriction of food intake. This type of surgical procedure seeks to alter the size (volume) of the stomach, therefore limiting the amount of food it can hold. The result is a premature feeling of satiety and a reduced intake of calories. Common examples of procedures producing food intake restriction include: vertical banded gastroplasty; gastric banding; and laparoscopic gastric banding. Through malabsorption, food intake restriction, or some combination of both, weight is reduced since less food either enters the stomach and/or less food remains in the small intestine long enough to be digested and absorbed.

In addition to surgery, devices and procedures have developed recent years which aim to:

(1) Restricting meal capacity and/or flow;

(2) Applying a barrier to digestion and/or absorption.

For example, devices and procedures which aim to restrict food influx into the stomach include banding devices, such as an adjustable gastroplasty ring and banding procedures (see U.S. Pub. No. 2004/0049209 A1 and 2004/0097989 A1 and U.S. Pat. No. 4,592,339); an implanted restrictor at the gastro-esophageal junction as see in WO 03/086246 A1 and WO 2004/064680 A1; and the positioning tool in WO 2004/064685 A1. Other devices aim to create an artificial distension signal in the stomach only by occupying space. Such as with balloons as in WO 02/35980 A2 and WO 2004/019765 A3. Other intragastric expanders are described in U.S. Pat. No. 6,675,809 B2 to Stack et al. and U.S. Pat. No. 5,868,141 A. Additionally, devices that stimulate the vagus nerve at the stomach may function by creating neural traffic simulating that invoked by distension, and thereby also constitutes an artificial distension signal (U.S. Pat. No. 6,587,719 B1 and U.S. Pat. No. 7,299,091 B2).

Although there have been many devices and procedures for weight control, it is necessary to invent a device that can control food intake quantitatively with feedback mechanism. The invention utilizes a basket made of a biocompatible material and embedded with a pressure sensor to restrict feeding and measure the pressure on the outer wall of the stomach in real time. Based on the pressure level, one can decide whether to continue or stop eating.

### SUMMARY

The present invention comprises a device and method for controlling body weight via a monitoring and feedback device for food intake utilizing MEMS pressure sensors embedded inside the basket material. The basket is custom made for each patient by 3D printing technology and is placed on the outer wall of the patient's stomach. The measured pressure value is integrated into a microcircuit and is transmitted to the wearable monitor via Bluetooth. When the patient eats too much, the pressure reaches the threshold level set by the doctor, and the monitor will immediately remind the obese person to stop eating. If the patient ignores the warning and continues to eat, the basket will reach its distensible limit, forcing a patient to stop taking food. The stomach is getting constrained gradually by the device during the feeding process, until the constrain reaches a threshold level to achieve the purpose of weight control.

### BRIEF DESCRIPTION OF THE DRAWINGS

FIG. 1 depicts the assembly diagram of the bariatric device of the present invention for restricting the food intake via net-shape basket.

FIG. 2 shows the placement of the basket structure.

FIG. 3 depicts the assembly diagram of the basket delivery system.

FIG. 4 depicts switch position 111 and 112 of the basket 110.

FIGS. 5A, 5B and 5C depict the fastening ways of the basket.

FIG. 6 shows the placement of the pressure sensor on the outside wall of gastric.

FIG. 7 shows the functional block diagram of the MEMS pressure sensor.

FIGS. 8, 9A and 9B shows the location of the sensor on the node of the basket.

FIGS. 10A and 10B depict the functional relationship between the volume and the pressure value.

FIG. 11 is a flow chart outlining the steps of an embodiment of the method of the present invention.

FIG. 12 shows the feeding and food emptying process of obese patients.

### DETAILED DESCRIPTION OF THE INVENTION

The following drawings are illustrative of particular embodiments of the present invention and therefore do not limit the scope of the invention. The drawings are not to scale (unless so stated) and are intended for use in conjunction with the explanations in the following detailed description. Embodiments will hereinafter be described in conjunction with the appended drawings wherein like numerals denote like elements.

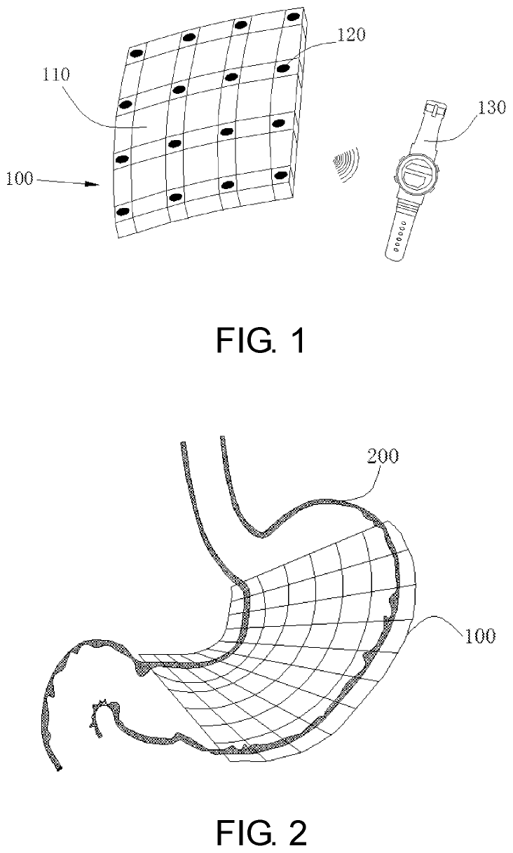

The present invention comprises a device and method for controlling body weight via a monitoring and feedback device for food intake. FIG. 1 depicts the detailed diagram of the implantable net-shaped basket system 100, including a basket 110, with several MEMS pressure sensors 120 embedded inside it. The sensor can measure the pressure of the stomach in real time. The pressure value is transmitted to the wearable monitor 130 via Bluetooth. The monitor 130 integrates these pressure signals and outputs a pressure-time curve.

Based on a physician's assessment of height, weight, the morphology of the stomach and energy consumption of obese patients, the basket 110 is custom made for each patient by 3D printing technology using biocompatible material, such as silicone material, PU (Polyurethane) etc. The material has good elasticity, it can surround the outer wall of the stomach well, even when the obese patients are in hungry condition, but won't affect gastric wriggle at the same time. The hardness, thickness and braid density has a lot effect on the physical properties of materials, and silicone materials with 10-40D (shore hardness), 0.2-2 mm in thickness and 10-35 PPI (pies per inch) are used in this invention.

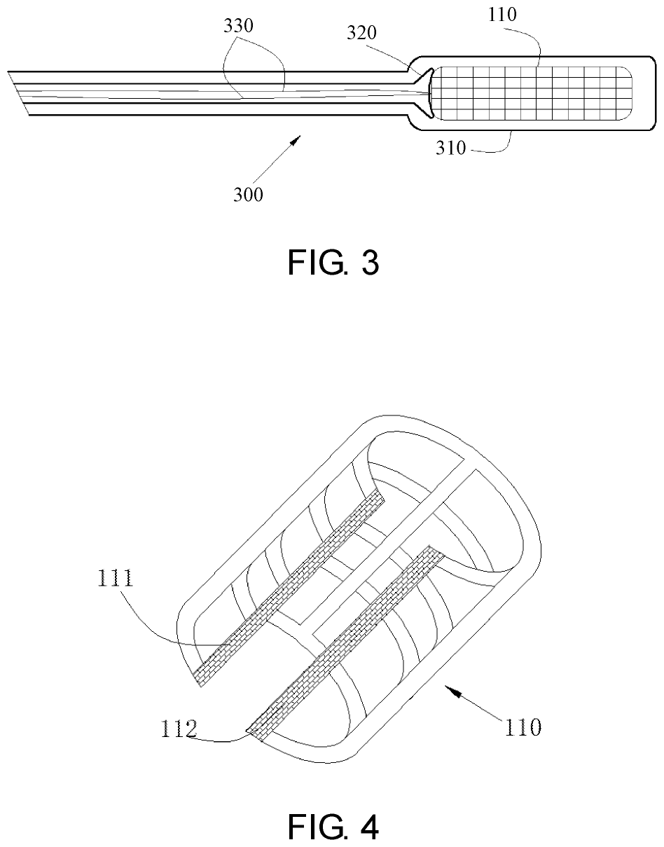

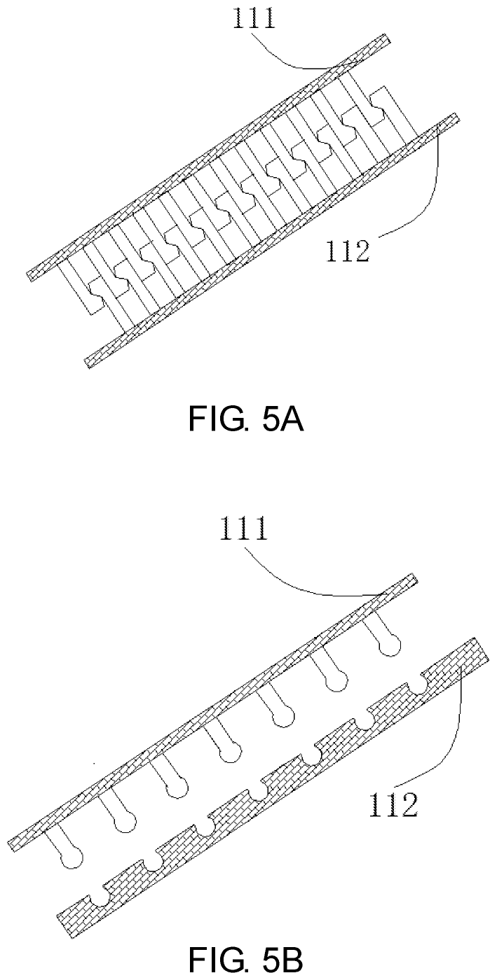

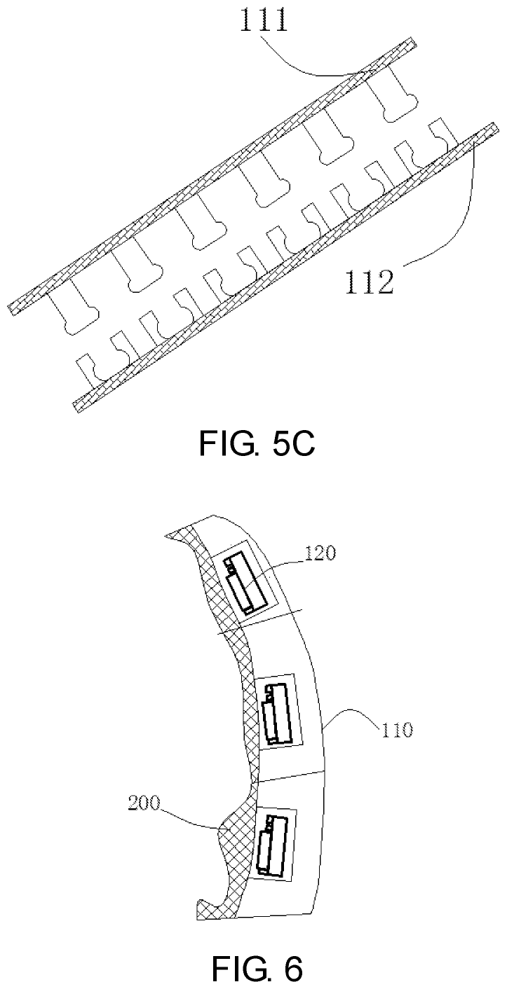

As shown in FIG. 2, the basket system 100 is placed on the outer wall of the patient's stomach by laparoscopic surgery (3 holes) via delivery system 300 (FIG. 3). Prior to implantation, the basket 110 is encased in a sheath 310, and the tether 330 is used to prevent it from slipping out. When placed near the greater curvature, the basket 110 is pushed out by the flared distal end 320 and expand to its original shape due to good elastic properties. Finally, it is fixed on the external wall of the stomach via two other instruments. FIG. 4 depicts switch position 111 and 112 of the basket 110. In one embodiment, the basket 110 is connected by means of a zipper after implantation. The switch position 111 and 112 ensure that the basket can be tightly wrapped around the outer wall of the stomach during the expansion and peristalsis of the stomach. In another embodiment, the fastening elements consist of a post portion and a receiving element as shown in FIGS. 5A-5C.

The MEMS pressure sensors 120 are embedded inside the basket material and placed on the side facing the stomach (see FIG. 6). These sensors can map the pressure change on the outside wall of the stomach in real time.

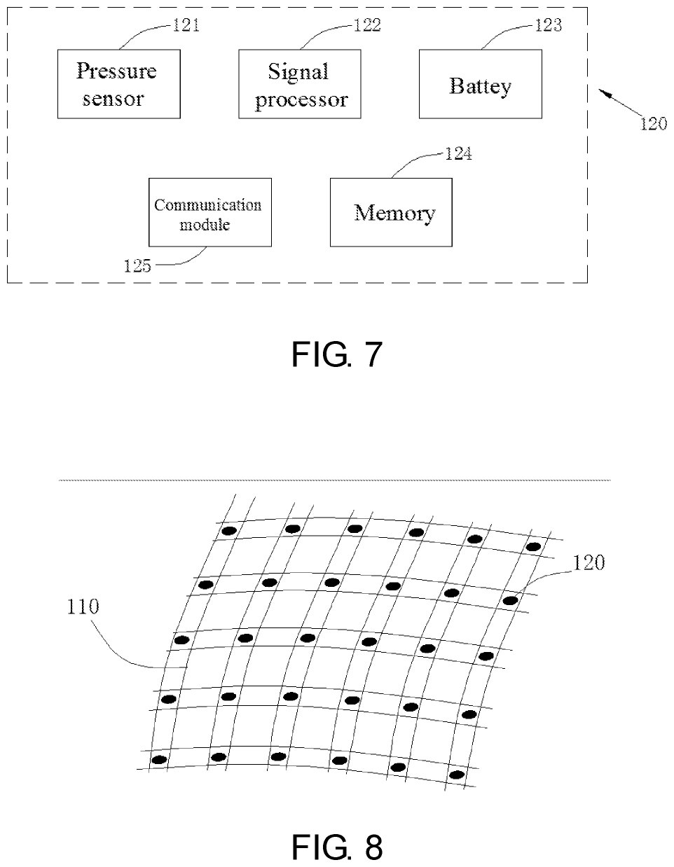

FIG. 7 shows the functional block diagram of the MEMS pressure sensor 120, including the pressure sensitive components 121, signal processor 122, battery 123, memory 124 and communication module 125. Pressure sensitive components 121 include at least one of a variety of different sensors, such as silicon (Si) piezoresistive sensor or silicon (Si) capacitive pressure sensor. The change in pressure converts into an electrical signal, and is amplified, calibrated, and temperature compensated by the signal processing module. Then, a series of intermediate signal processing such as filtering is performed, and the electrical signal is converted into a digital signal by an A/D converter, and finally the result is transmitted to a wearable monitor through the communication module 125.

Communication module 125 is designed to transmit the measured pressure value to other devices, such as wearable display 130, including any suitable hardware (e.g., antenna, Bluetooth), software, or any combination thereof. Batteries 123, for example, can be rechargeable or non-rechargeable. Memory 124 may comprise any volatile, non-volatile, magnetic or electrical medium such as RAM, ROM, NVRAM, EEPROM, flash memory, or any other digital medium.

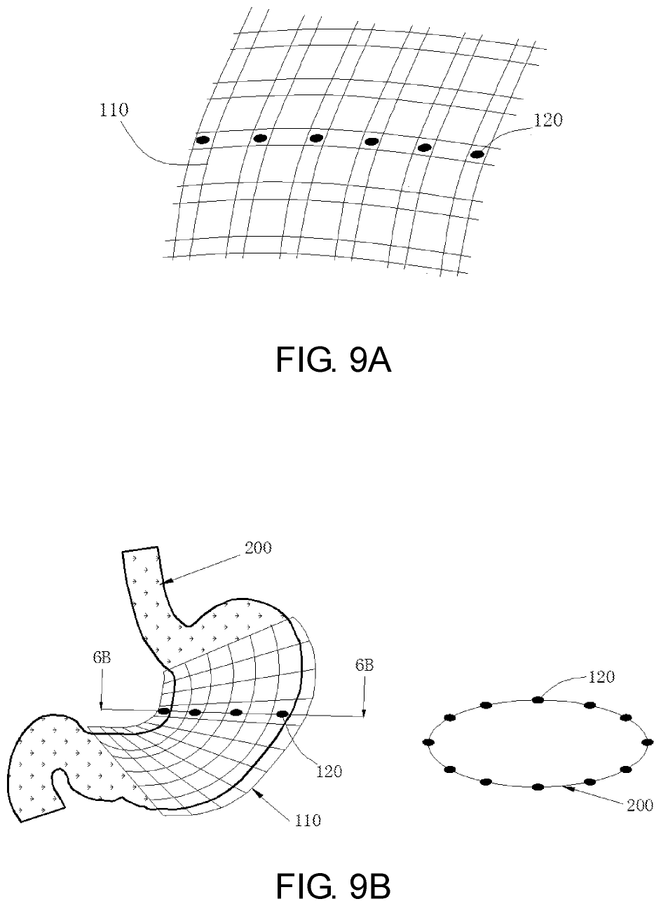

In one embodiment, the sensors 120 locate on each node of the basket 110, the number ranging from 20 to 200 (see FIG. 8). The sides of the sensors 120 are 1-5 mm in length, its thickness is 0.1-1 mm. The monitor 130 integrates these pressure signals and outputs a pressure-time curve. In other embodiment, the sensors 120 are placed at some perimeter around the outer wall of the stomach (see FIGS. 9A and 9B).

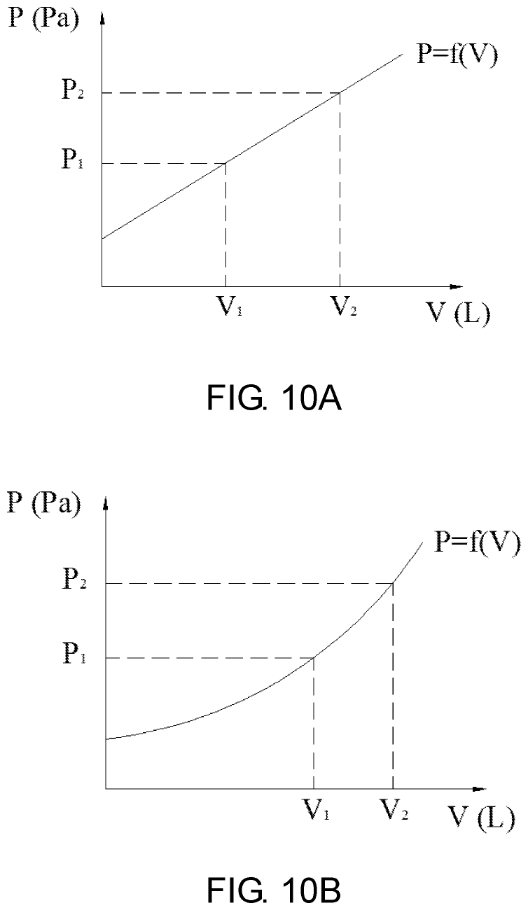

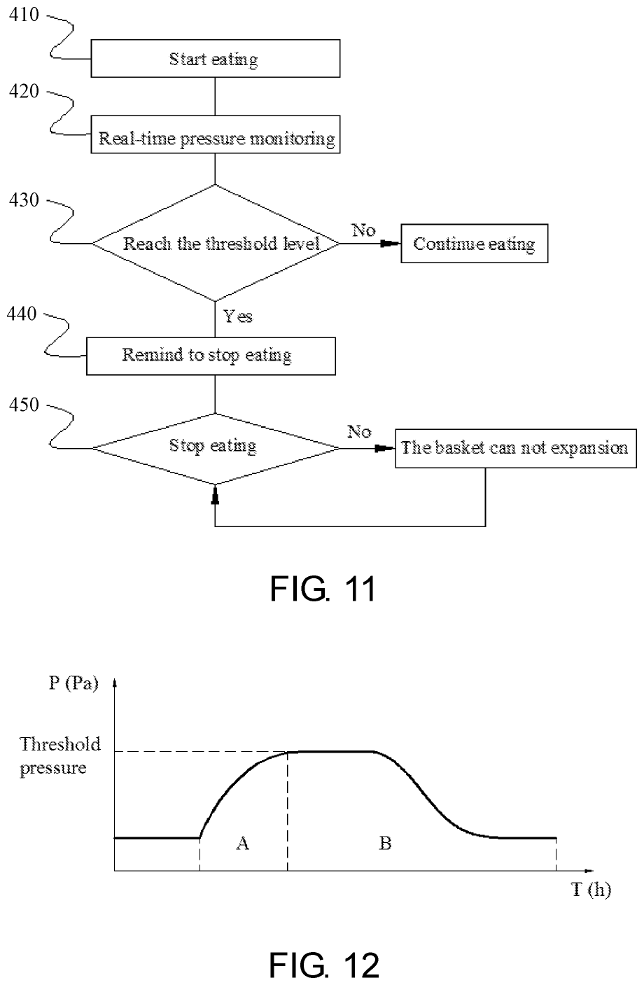

The volume of the stomach gradually expands during the feeding process, so the basket 110 is gradually stretched, and the pressure value measured by the sensors increases accordingly until the feeding stops. Due to the inherent physical properties of the material, such as elastic modulus, expansion coefficient, etc., there is a certain functional relationship between the volume and stretching force (or the pressure value) during the process of the basket being stretched by the stomach eating. The function relationship is as shown in FIG. 10A, probably FIG. 10B. The doctor will customize the food intake (in terms of volume) according to the weight loss plan of the obese patient, so that there will be a corresponding pressure threshold P, or a range of pressure values (P1-P2). When the obese patient eats too much, the sensor 120 will be subjected to greater pressure and reaches the threshold level set by the doctor. The wearable monitor 130 will sound an alarm to remind the obese person to stop eating. If the patient ignores the warning and continues to eat, the basket will reach its distensible limit, forcing a patient to stop taking food (see FIG. 11).

FIG. 12 shows the pressure-time curve of the stomach of obese patients, through which the feeding (Process A) and food emptying process (Process B) can be observed via the wearable monitor 130. Not only food intake of obese patients can be controlled quantitatively via the basket system, but also the gastric motility, gastroparesis, such as delayed gastric emptying or acceleration of gastric emptying, can be diagnosed according to the pressure-time curve of Process B. Generally speaking, it usually takes 4-6 hours for the stomach to completely empty, though different food has different emptying speed, which is related to the physical and chemical composition of food. If process B lasts too long or too short, the patient's gastric emptying can be judged combined with the patient's recent diet.

What is claimed is:

1. An implantable device for controlling weight of a body comprising:
a net-shaped basket being placed on an outer wall of a stomach of a patient to restrict food intake;
several MEMS pressure sensors being embedded inside the basket material to measurea pressure change of the stomach of the patient;
a wearable monitor.

2. The bariatric device as claimed in claim 1, wherein the basket is custom made for the patient by 3D printing technology, based on a physician's assessment of height, weight, and energy consumption of the patient.

3. The bariatric device as claimed in claim 1, wherein the basket is made of biocompatible material, such as silicone material, PU etc.

4. The bariatric device as claimed in claim 1, wherein the basket is characterized by certain hardness, thickness and braid density.

5. The bariatric device as claimed in claim 1, wherein the basket is implanted by a delivery system via minimally invasive procedures, such as laparoscopic surgery.

6. The bariatric device as claimed in claim 1, wherein the basket is connected after implantation by certain structures, such as a zipper, a post portion and a receiving element.

7. The bariatric device as claimed in claim 1, wherein the MEMS pressure sensors are embedded inside the basket material and placed on a side facing the stomach of the patient.

8. The bariatric device as claimed in claim 1, wherein the MEMS pressure sensors locate on each node of the basket or are placed at some perimeter around the outer wall of the stomach of the patient.

9. The bariatric device as claimed in claim 1, wherein the MEMS pressure sensors measure the pressure changes in the outer wall of the stomach of the patient in real time.

10. The bariatric device as claimed in claim 1, wherein the MEMS pressure sensors comprise pressure sensitive components, a signal processor, a battery, a memory and a communication module.

11. The bariatric device as claimed in claim 8, wherein the pressure sensitive components further comprise at least one of a variety of different sensors, such as a Si piezoresistive sensor or a Si capacitive pressure sensor.

12. The bariatric device as claimed in claim 1, wherein the MEMS pressure sensors transmits a measured pressure value to other devices, such as a wearable display.

13. The bariatric device as claimed in claim 1, wherein the wearable monitor communicates with pressure sensors implanted in the body.

14. The bariatric device as claimed in claim 1, wherein the wearable monitor outputs a pressure-time curve of the stomach of the patient.

15. The bariatric device as claimed in claim 1, wherein monitor reminds the patient to stop eating when a measured pressure reaches a threshold level set by a doctor.

16. The bariatric device as claimed in claim 14, wherein the pressure-time curve of the stomach is used to diagnose the food emptying process.
